(19) United States
(12) Patent Application Publication
(10) Pub. No.: US 2024/0285909 A1
(43) Pub. Date: Aug. 29, 2024

(54) Catheter Component Securement Apparatus and Associated System

(71) Applicant: Becton, Dickinson and Company, Franklin Lakes, NJ (US)

(72) Inventors: Sudev GS, Kozhikode (IN); Priya Saraf, Bengaluru (IN)

(21) Appl. No.: 18/115,189

(22) Filed: Feb. 28, 2023

(51) Int. Cl. A61M 25/02

(52) U.S. Cl. CPC A61M 25/02; A61M 2025/024; A61M 2025/0253

(57) ABSTRACT

An apparatus for removably securing a medical device to a patient includes a flexible structure having a top surface and a bottom surface and a securement structure coupled to the top surface of the flexible structure, the securement structure configured to receive the medical device. The securement structure includes a base and a cover movably coupled to the base, wherein the cover is movable between at least a first position and a second position, wherein the cover and the base are engaged with the medical device when the cover is in the second position.

### BACKGROUND OF THE INVENTION

### Field of the Disclosure

The present disclosure relates generally to a securement apparatus for catheter components.

### Description of Related Art

Currently, patients requiring catheterization over an extended period during their treatment may need their catheter to be secured to maintain the position of the catheter line and to prevent accidental dislodgement. There are many ways to secure the catheter lines to the patient body. The most common method is taping the catheter to the patient. But there are drawbacks associated with this method. Drawbacks include increased risk of contaminant accumulation at the tape area, and increased risk of unwanted and/or accidental movement of catheter while removing the adhesive tape. The tapes require frequent changes, which often leads to skin irritation and/or damage. Further, using tape typically results in adhesive residue on the catheter body which attracts contaminations. Even further, the process of attaching and detaching tape over the catheter is a tedious task to the caregiver and causes discomfort to the patient.

Catheter stabilization devices address many of the drawbacks of the taping method. They offer more effective way of securement of catheter. Most of these devices include a plastic lock/clamp to secure the catheter both longitudinal and lateral movement. These are stuck to the patient with the adhesive layer provided on the bottom surface. Drawbacks to existing catheter stabilization devices include usability of installing and removal of the device, as there are multiple steps required for complete securement of catheters. Another drawback is that intravenous (IV) catheter hubs and wings come in varied designs and dimensions. Existing stabilization devices are designed to be used for very specific types of catheters and thus lack adaptability to different types of catheter designs. A further drawback is increased risk of accidental dislodgement of the catheter while removing the IV dressing.

Accordingly, those skilled in the art continue with research and development efforts in the field of catheter component securement apparatuses and systems for securing catheter components.

### SUMMARY OF THE INVENTION

Disclosed is an apparatus for removably securing a medical device to a patient.

In one example, the apparatus includes a flexible structure having a top surface and a bottom surface and a securement structure coupled to the top surface of the flexible structure, the securement structure configured to receive the medical device. The securement structure includes a base and a cover movably coupled to the base, wherein the cover is movable between at least a first position and a second position, wherein the cover and the base are engaged with the medical device when the cover is in the second position.

The apparatus may further include a peel tab defined by the flexible structure and an orientation indicator defined on the top surface of the flexible structure. The bottom surface may include an adhesive.

The securement structure may be a clamp. The securement structure may include a base having at least one engagement portion configured to align with the medical device. The engagement portion may be one or more of a recessed portion, a base alignment structure, a top alignment structure, a stabilizing structure, a clip, a wing recess, and a foam pad.

The base may define at least one recessed portion configured to nest with the medical device. The base may define at least one wing recess configured to nest with a wing of the medical device. The base may define at least one ledge. The base may include at least one base alignment structure. The base alignment structure may be a pin.

The securement structure may further include a cover coupled to the base. The cover may be hingedly coupled to the base. The base may include a foam pad and the cover may include a second foam pad. One or more of the foam pad and the second foam pad may be memory foam. One or more of the foam pad and the second foam pad may define a recessed portion.

The cover may include at least one top alignment structure. The cover may include a stabilizing structure. The stabilizing structure may be a rubber gripper.

The base may include a flanged portion and the cover may include a lip portion configured to removably lock with the flanged portion. The cover may be movable between at least a first position and a second position, and when the cover is in the second position, the flanged portion and the lip portion may snap together to form a clamp.

The securement structure may include a first receiving sleeve fixedly coupled to the base and a second receiving sleeve opposed from the first receiving sleeve and movably coupled to the base. The second receiving sleeve may be movably coupled to the base with a spring loaded track. The securement structure may include a tab coupled to the second receiving sleeve.

In another example, the apparatus includes a flexible structure 110 having a top surface 112 and a bottom surface 114 and a securement structure 120 coupled to the top surface 112 of the flexible structure 110. The securement structure 120 is configured to receive the medical device 50. The securement structure includes a base 130, a first receiving sleeve 160 fixedly coupled to the base 130, and a second receiving sleeve 162 opposed from the first receiving sleeve 160 and movably coupled to the base 130.

Also disclosed is a system for removably securing a medical device to a patient.

In one example, the system includes a medical device and an apparatus removably coupleable with the medical device, the apparatus includes a flexible structure having a top surface and a bottom surface and a securement structure coupled to the top surface of the base, the securement structure configured to receive the medical device.

The medical device of the system may be a portion of a catheter.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective schematic view of an apparatus for securing a medical device to a patient;

FIG. 2 is a cross-sectional side view of the apparatus of FIG. 1;

FIG. 3 is a perspective schematic view of an apparatus for securing a medical device to a patient;

FIG. 4 is a perspective schematic view of a system for securing a medical device to a patient;

FIG. 5 is a perspective schematic view of the system of FIG. 4;

FIG. 6 is a perspective schematic of an apparatus for securing a medical device to a patient;

FIG. 7 is a perspective schematic view of a system for securing a medical device to a patient;

FIG. 8 is a perspective schematic view of the system of FIG. 7;

FIG. 9 is a perspective schematic view of an apparatus for securing a medical device to a patient;

FIG. 10 is a perspective schematic view of a system for securing a medical device to a patient;

FIG. 11 is a perspective schematic view of the system of FIG. 10;

FIG. 12 is a perspective schematic of an apparatus for securing a medical device to a patient;

FIG. 13 is a cross-sectional side view of the apparatus of FIG. 12;

FIG. 14 is a perspective schematic view of a system for securing a medical device to a patient;

FIG. 15 is a perspective schematic view of the system of FIG. 14;

FIG. 16 is a perspective schematic of an apparatus for securing a medical device to a patient;

FIG. 17 is a perspective schematic view of a system for securing a medical device to a patient; and

FIG. 18 is a perspective schematic view of the system of FIG. 17.

### DETAILED DESCRIPTION OF THE DISCLOSURE

Spatial or directional terms, such as “left”, “right”, “inner”, “outer”, “above”, “below”, and the like, are not to be considered as limiting as the disclosure can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

Reference herein to “one or more examples” means that one or more feature, structure, or characteristic described in connection with the example is included in at least one implementation. The phrase “one or more examples” in various places in the specification may or may not be referring to the same example.

As used herein, a system, apparatus, structure, article, clement, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The disclosure is an improved device for effective securement of IV catheters. It consists of an adhesive patch to attach the device to a patient's skin and a plastic feature on the middle of the adhesive pad for securement of a catheter or catheter component.

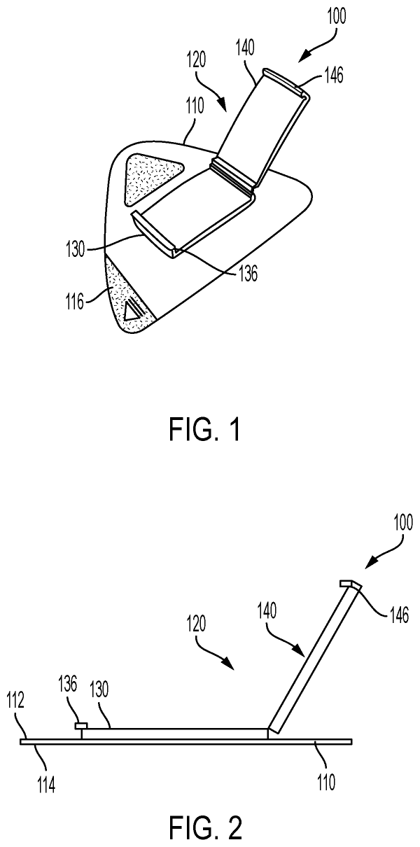

Referring to FIG. 1, disclosed is an apparatus 100 for removably securing a medical device 50 to a patient. The apparatus 100 may be designed for removably securing and stabilizing a medical device 50, such as a catheter or component of a catheter, to a patient. The apparatus 100 may be removably secured to the patient in that it can stick to a patient's body via an adhesive or other means of attachment without becoming permanently bonded to the patient. The disclosed apparatus 100 may be generally defined by having an attachment portion (flexible structure 110 described below) for coupling the apparatus 100 with a patient and a securing portion (securement structure 120 described below) for receiving the medical device 50 and securing the medical device 50 to the apparatus 100. The various examples below describe optional features that may be individually or collectively included on the apparatus 100 to accommodate any medical device 50.

Referring to FIG. 2, in one or more example, the apparatus 100 includes a flexible structure 110 having a top surface 112 and a bottom surface 114. The flexible structure 110 is flexible in that it is configured to conform to a patient for even and secure coupling. The flexible structure 110 may be of any shape and geometry without limiting the scope of the disclosure. For example, as shown in FIG. 1, the flexible structure 110 may have a generally triangular shape. In another example, as shown in FIG. 16, the flexible structure 110 may have a generally trapezoidal shape. It is contemplated that the flexible structure 110 may have any other shape, such as generally round or rectangular, without limiting the scope of the disclosure. The flexible structure 110 may comprise any suitable material for conforming to a patient, such as a polymeric material. In one example, the bottom surface 114 includes an adhesive.

The bottom surface 114 may include a protective film 114a removably coupled and peelable from the bottom surface 114. For example, when an adhesive is evenly distributed across the bottom surface 114 for attachment to a patient, the bottom surface 114 may include the protective film 114a, such as peclable sheet, to keep the bottom surface 114 clean and protected from adhering to other objects prior to coupling with a patient.

In one example, the apparatus 100 includes at least one peel tab 116 defined by the flexible structure 110. The peel tab 116 may be defined by a crease, cut, perforation, or any other means of defining a removable portion that is coupled with the protective film 114a to facilitate removal of the protective film 114a when securing the apparatus 100 on a patient.

Referring to FIG. 1, in one or more examples, the apparatus 100 may further include an orientation indicator 118 defined on the top surface 112. The orientation indicator 118 may be a virtual indicator, such as a highlighted portion of the flexible structure 110, to facilitate proper orientation of the apparatus 100 on a patient.

Referring back to FIG. 1 and FIG. 2, the apparatus 100 includes a securement structure 120 coupled to the top surface 112 of the flexible structure 110. The securement structure 120 is configured to receive the medical device 50. For example, the securement structure 120 may be shaped and sized to match the geometry of the medical device 50 or portions of the medical device 50 so that the medical device 50 can securely nest within the securement structure 120.

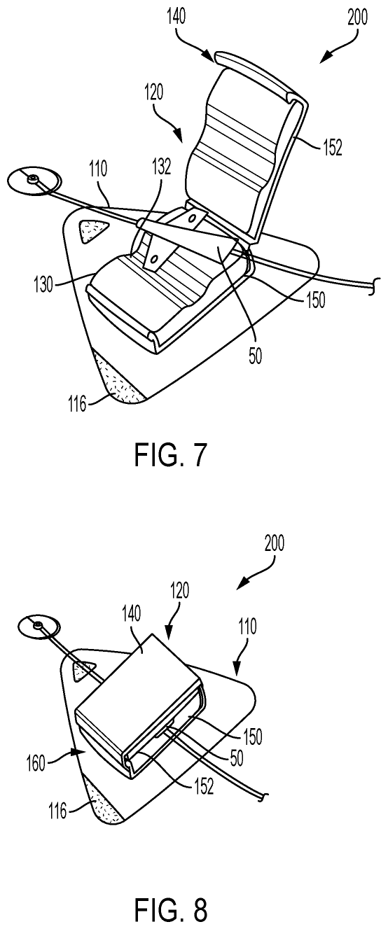

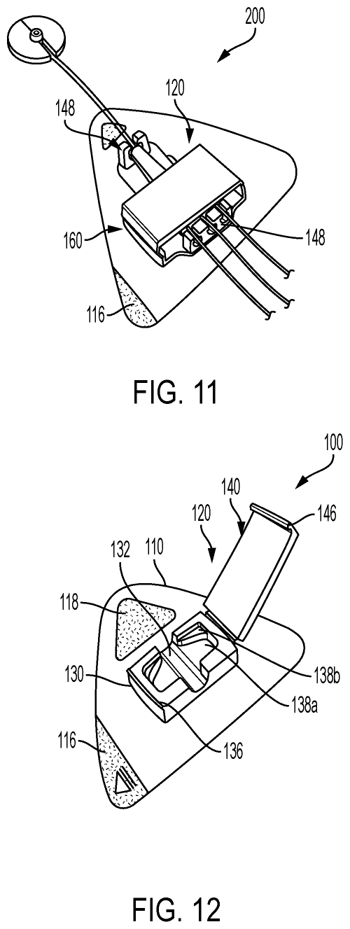

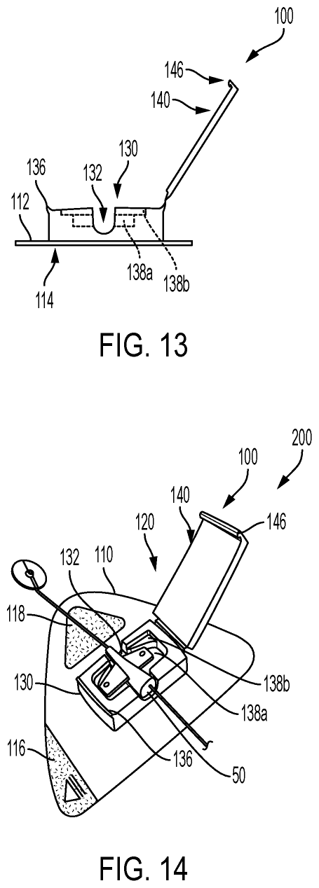

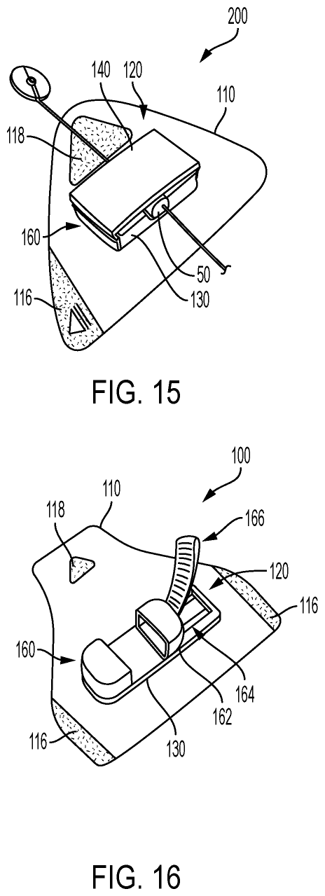

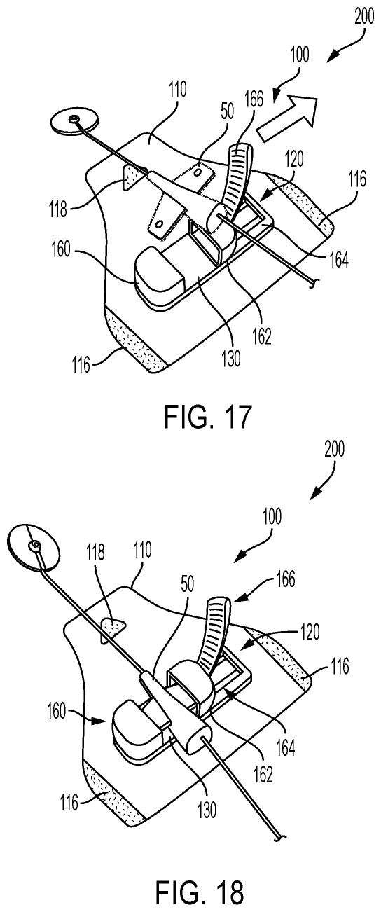

Referring generally to FIGS. 1-18, the securement structure 120 comprises a base 130. The base 130 may include at least one engagement portion 122 configured to align with the medical device 50. The engagement portion 122 may be any feature, such as a recessed or protruding portion, sized and shaped to nest with at least a portion of the medical device 50. In one example, the engagement portion 122 is one or more of a recessed portion 132, a base alignment structure 134, a top alignment structure 144, a stabilizing structure 142, a clip 148, a wing recess 138a, and a foam pad 150. Further, the engagement portion 122 may be generally defined by the base 130 which may have any means for removably coupling with the medical device 50. For example, the base 130 may include a gripping or adhesive material configured to secure the medical device 50 in place.

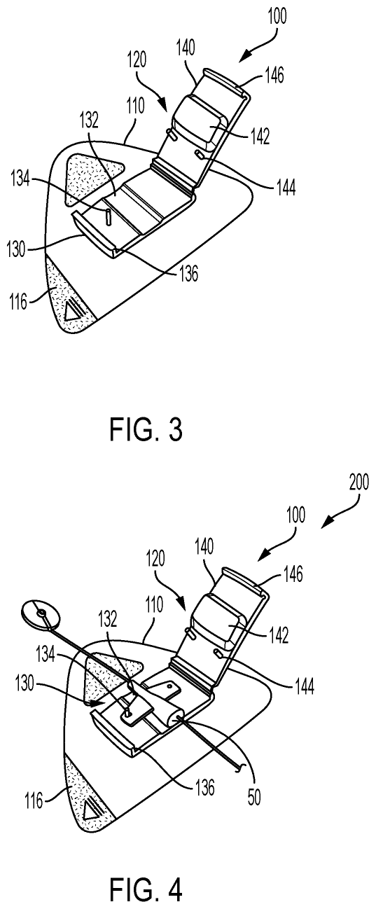

Referring to FIG. 3, in one or more examples, the base 130 may define at least one recessed portion 132 configured to nest with the medical device 50. The recessed portion 132 may be generally rounded or rectangular based upon the shape of the medical device 50.

Referring generally to FIGS. 3-5, the securement structure 120 may include one locator pin on the base 130 which can receive the catheter. Locator pin restricts the lateral movement of the catheter. The catheter is sandwiched between the cover 140 and base 130 of the securement structure 120, and a snap mechanism is used to lock the securement structure 120. There are two more pins in the cover 140 of the securement structure 120 which restrict lateral motion of the catheter. A silicone rubber gripper reduces vertical movement. The disclosure demonstrates following features: 1) it includes a single flap design that simplified installing and removing; 2) it is adaptable to varied catheter designs; and 3) the cover 140 prevents catheter dislodgement during removal.

Referring to FIGS. 3 and 4, in one or more examples, the base 130 may include at least one base alignment structure 134. In one example, the base alignment structure 134 is a pin.

Referring to FIGS. 3-5, in one or more example, the cover 140 may include a stabilizing structure 142. The stabilizing structure 142 may be configured to engage with the medical device 50 when the cover 140 is in a closed position and may function to improve stabilization of the medical device 50, such as limiting lateral and vertical movement within the securement structure 120. In one example, the stabilizing structure 142 is a rubber gripper.

Still referring to FIGS. 3 and 4, in one or more example, the cover 140 may include at least one top alignment structure 144. As illustrated in FIGS. 3 and 4, the cover 140 may include two or more alignment structure 144. The alignment structure 144 serves as additional alignment support and stabilization of the medical device 50 within the securement structure 120. The alignment structure 144 may be positioned on the cover 140 to enable engagement with a feature of the medical device 50, such as a hole sized and shaped to receive the alignment structure 144, locking the medical device 50 in a predetermined position. In another example, the alignment structure 144 may be positioned on the cover 140 such that it abuts the medical device 50, limiting and/or preventing movement of the medical device 50 within the securement structure 120.

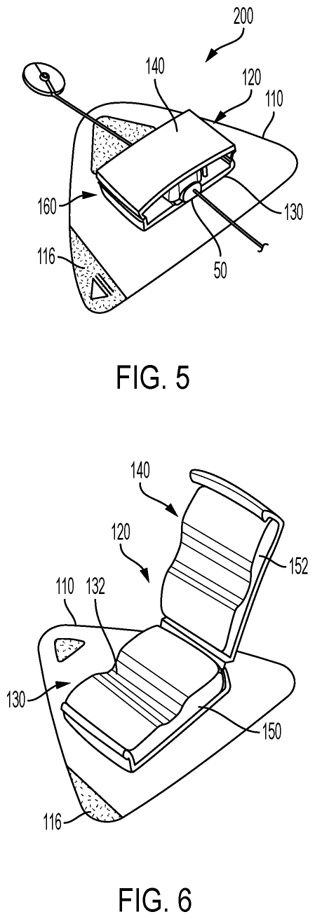

Referring to FIGS. 1-15, the securement structure 120 may further include a cover 140 coupled to the base 130. The cover 140 may be hingedly coupled to the base 130 to facilitate movement of the cover 140 between at least a first position, see FIG. 4, wherein the cover 140 is in an open position, and a second position, see FIG. 5, wherein the cover 140 is a in closed and locked position. The cover 140 of the securement structure 120 may be a clamp 160 such that it is configured to clamp around the medical device 50 to secure it in place. For example, the base 130 may define a flanged portion 136 and the cover 140 may define a lip portion 146 configured to removably lock with the flanged portion 136, thus forming a clamp. Specifically, the cover 140 in this example is movable between at least a first position and a second position, and when the cover 140 is in the second position, the flanged portion 136 and the lip portion 146 snap together to form a clamp 160.

Referring to FIGS. 6-8, the disclosure may utilize a simple and unique mechanism for securing the catheter. In this example, securement structure 120 includes a higher density memory foam for securing the medical device 50, such as a catheter. Memory foam is ideal for adapting to the shape of the catheter design and holding it in place. Movement of catheter will be restricted in all directions as memory foam can occupy all negative spaces and keep the catheter in place.

The disclosure demonstrates the following advantages: 1) it is easy to use and has intuitive design; 2) requires few workflow steps; 3 utilizes a single flap design simplifies installation and removal; 4) adaptable to various catheter designs; and 5) prevents catheter dislodgement during removal.

Still referring to FIGS. 6-8, in one or more examples, the base 130 may include a foam pad 150 and the cover 140 may include a second foam pad 152. The foam pad 150 and second foam pad 152 are configured to facilitate stabilization of the medical device 150 between the bottom portion 130 and the cover 140. In one example, one or more of the foam pad 150 and the second foam pad 152 is comprised of memory foam. In another example, one or more of the foam pad 150 and the second foam pad 152 defines a recessed portion 132.

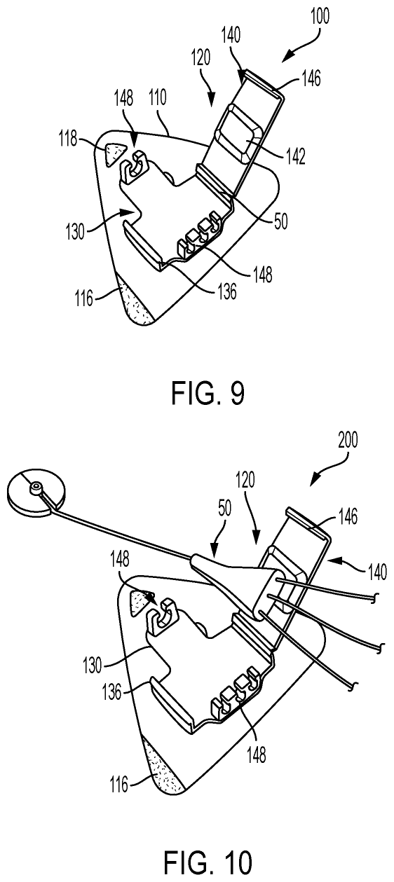

Referring generally to FIGS. 9-11, securement structure 120 includes a clamp design with a living hinge. The base 130 includes at least one silicon clip 148 to lock the medical device 50, and more specifically lumens of the medical device 50. The securement structure 120 may also include a stabilizing structure 142, such as a silicone pad, to create friction and hold the medical device 50, such as a catheter hub, tightly against the base 130 when in a closed configuration. Because this locking mechanism is independent of wing and hub design, it is adaptable to different types of catheters. As shown in FIGS. 9 and 10, the securement structure 120 may more than one stabilizing structure 142 and each stabilizing structure 142 may be located at a different location on the base 130 to stabilize different portions of the medical device 50.

This disclosed design advantageously restricts the movement of the catheter as both the lumen ends are arrested in the silicone snaps. The disclosed demonstrates following features: 1) utilizes at least one silicon clip 148 snap feature that is adaptable to different types of catheters; 2) utilizes a single flap design that improves usability during installation and removal; and 3) the cover 140 prevents catheter dislodgement while removal.

Referring generally to FIGS. 12-15, in one or more examples, base 130 includes a depression/pocket to contain a medical device 50, and more specifically hub and wings of the catheter. This pocket is sized and shaped with enough clearance to accommodate different catheter types and designs. Once closed, the securement structure 120 is configured to secure the catheter from undergoing longitudinal movement. The combined locking feature safely secures the catheter without permitting longitudinal or lateral movements. The number of steps required to secure the catheter is simplified compared to existing stabilization devices.

The disclosure demonstrates following advantages: 1) the securement pocket design is adaptable to different types of catheters; 2) the design allows simplified installation and removal; 3) the single flap design improves usability during installation and removal; and 4) the cover 140 prevents catheter dislodgement during removal.

Still referring to FIGS. 12-15, in one or more examples, the base 130 may define at least one wing recess 138a configured to nest with a wing 52 of the medical device 50. As shown in FIG. 12, the base 130 may define two or more wing recess 138a. The base 130 may further define at least one ledge 138b, see FIG. 14.

Referring generally to FIGS. 16-18, the securement structure 120 includes a slide and lock slot feature with a peel tab to lock the catheter. The slots clearance is designed in such a way that it can accommodate different types of catheter hubs and wings. The locking of catheter is achieved by a spring-loaded slot feature. The user pulls this feature with the help of the pull tag and place the catheter hub and wings inside the securement slot to lock the lateral and longitudinal movement of the catheter. The number of steps required to secure the catheter is much less compared to existing stabilization devices. The disclosure in this example demonstrates following features: 1) it offers an adaptable slot securement feature which can be used on different types of catheter; 2) it allows easier installation and removal with a smaller number of steps; 3) it utilizes a pull tag to improve usability while installation and removal; and 4) it utilizes a unique spring-loaded securement mechanism for adaptable fit. This allows for it to be adaptable to different types of catheters.

Still referring to FIGS. 16-18, the securement structure 120 may include a first receiving sleeve 160 fixedly coupled to the base 130 and a second receiving sleeve 162 opposed from the first receiving sleeve 160. The second receiving sleeve 162 may be movably coupled to the base 130. For example, the second receiving sleeve 162 may movably coupled to the base 130 with a spring loaded track 164 such that the second receiving sleeve 162 may move along the spring loaded track 164 to allow for insertion and removal of the medical device 50 within the securement structure 120. The securement structure 120 may further include a pull tab 166 coupled to the second receiving sleeve 162. The pull tab 166 is configured to facilitate movement of the second receiving sleeve 162 along the spring loaded track 164 for placement and removal of the medical device 50.

In one example, the apparatus 100 for removably securing a medical device 50 to a patient includes a flexible structure 110 having a top surface 112 and a bottom surface 114, and a securement structure 120 coupled to the top surface 112 of the flexible structure 110. The securement structure 120 is configured to receive the medical device 50. The securement structure includes a base 130, a first receiving sleeve 160 fixedly coupled to the base 130, and a second receiving sleeve 162 opposed from the first receiving sleeve 160 and movably coupled to the base 130.

In one example, the second receiving sleeve 162 is movably coupled to the base 130 with a sliding track 164. In another example, wherein sliding track 164 is spring loaded. In one example, the second receiving sleeve 162 is movable between at least a first position and a second position, the first position spaced a distance from the first receiving sleeve 160 that is greater than a width of the medical device 50. Therefore, in the first position, the second receiving sleeve 162 is in an open position to facilitate placement of the medical device 50 in the securement structure 120. Further, when the second receiving sleeve 162 is in the second position, it is closed such that it is securely engaged with the medical device 50 to reduce and/or eliminate movement of the medical device 50.

Referring generally to FIGS. 4, 5, 7, 8, 10, 11, 14, 15, 17, and 18, disclosed is a system 200 for removably securing a medical device 50 to a patient. The system 200 includes a medical device 50 as described herein. In one example, the medical device 50 is a catheter or a portion of a catheter, such as a hub, IV line, or catheter tubing.

The system 200 further includes an apparatus 100, as shown and described herein, removably coupleable with the medical device 50. The apparatus 100 includes a flexible structure 110 having a top surface 112 and a bottom surface 114. The apparatus 100 further includes a securement structure 120 coupled to the top surface 112 of the flexible structure 110, the securement structure 120 configured to receive the medical device 50.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. An apparatus removably securing a medical device to a patient, the apparatus comprising:
a flexible structure having a top surface and a bottom surface; and
a securement structure coupled to the top surface of the flexible structure, the securement structure configured to receive the medical device, the securement structure comprising:
a base; and
a cover; movably coupled to the base, wherein the cover is configured to move between at least a first position and a second position, wherein the cover and the base are engaged with the medical device when the cover is in the second position.

2. The apparatus of claim 1, further comprising a peel tab defined by the flexible structure.

3. The apparatus of claim 1, further comprising an orientation indicator defined on the top surface.

4. The apparatus of claim 1, wherein the bottom surface comprises an adhesive.

5. The apparatus of claim 1, wherein the securement structure is a clamp.

6. The apparatus of claim 1, wherein the base comprises at least one engagement portion configured to align with the medical device.

7. The apparatus of claim 6, wherein the engagement portion is one or more of a recessed portion, a base alignment structure, a top alignment structure, a stabilizing structure, a clip, a wing recess, and a foam pad.

8. The apparatus of claim 1, wherein the base defines at least one recessed portion configured to nest with the medical device.

9. The apparatus of claim 1, wherein the base defines at least one wing recess configured to nest with a wing of the medical device

10. The apparatus of claim 1, wherein the base defines at least one ledge.

11. The apparatus of claim 1, wherein the base comprises at least one base alignment structure.

12. The apparatus of claim 11, wherein the base alignment structure is a pin.

13. The apparatus of claim 1, wherein the cover is hingedly coupled to the base.

14. The apparatus of claim 1, wherein the cover is pivotally coupled to the base.

15. The apparatus of claim 1, wherein the base comprises a foam pad and the cover comprises a second foam pad.

16. The apparatus of claim 15, wherein one or more of the foam pad and the second foam pad is memory foam.

17. The apparatus of claim 15, wherein one or more of the foam pad and the second foam pad defines a recessed portion.

18. The apparatus of claim 1, wherein the cover comprises at least one top alignment structure.

19. The apparatus of claim 1, wherein the cover comprises a stabilizing structure.

20. The apparatus of claim 19, wherein the stabilizing structure is a rubber gripper.

21. The apparatus of claim 1, wherein:
the base comprises a flanged portion; and
the cover comprises a lip portion configured to removably lock with the flanged portion.

22. The apparatus of claim 21, wherein, when the cover is in the second position, the flanged portion and the lip portion snap together to form a clamp.

23. An apparatus for removably securing a medical device to a patient, the apparatus; comprising:
a flexible structure having a top surface and a bottom surface; and
a securement structure coupled to the top surface of the flexible structure, the securement structure configured to receive the medical device, the securement structure comprising:
a base;
a first receiving sleeve fixedly coupled to the base; and
a second receiving sleeve opposed from the first receiving sleeve and movably coupled to the base.

24. The apparatus of claim 23, wherein the second receiving sleeve is movably coupled to the base with a sliding track.

25. The apparatus of claim 24, wherein the sliding track is spring loaded.

26. The apparatus of claim 23, wherein the second receiving sleeve is movable between at least a first position and a second position, the first position spaced a distance from the first receiving sleeve that is greater than a width of the medical device.

27. The apparatus of claim 23, further comprising a pull tab coupled to the second receiving sleeve.

28. A system for removably securing a medical device to a patient, the system comprising:
a medical device; and
an apparatus removably coupleable with the medical device, the apparatus comprising:
a flexible structure having a top surface and a bottom surface; and
a securement structure coupled to the top surface of the base, the securement structure configured to receive the medical device.

29. The system of claim 28, wherein the medical device is a portion of a catheter.
